(19) United States
(12) Patent Application Publication
(10) Pub. No.: US 2014/0176904 A1
(43) Pub. Date: Jun. 26, 2014

(54) Ophthalmic Aberrometer Capable of Subjective Refraction

(71) Applicant: Ming Lai, Dublin, CA (US)

(72) Inventor: Ming Lai, Dublin, CA (US)

(21) Appl. No.: 14/076,276

(22) Filed: Nov. 11, 2013

Related U.S. Application Data
(60) Provisional application No. 61/724,910, filed on Nov. 10, 2012.

(51) Int. Cl. A61B 3/103

(52) U.S. Cl. CPC A61B 3/1035
USPC 351/206; 351/246

(57) ABSTRACT

The present invention contemplates an ophthalmic aberrometer combining measurements of wavefront aberrations and subjective refraction into a single instrument and refers both measurements to the same corneal plane The present invention also contemplates an ophthalmic aberrometer employing an open field and subjective correction to overcome instrument myopia and to ensure accurate measurement of the best-corrected visual acuity in addition to measurement of wavefront aberrations. The present invention further contemplates an ophthalmic aberrometer implementing an optical relay with adjustable optical power compensation to eliminate the need for flipping plurality sets of trial lenses for defocus correction. The present invention also further contemplates an ophthalmic aberrometer making wavefront measurement along a viewing path of the subject eye and enabling accurate measurement of the residual wavefront aberrations after compensating for the subjective refraction.

This application claims the benefit of U.S. Provisional Application No. 61/724,910, filed on Nov. 10, 2012.

### 1. RELATED FIELD

The invention relates to a method and device for measuring optical aberrations and refractive errors of a human eye. In particular, the invention relates to a method and device for measuring optical aberrations and refractive errors of a human eye via a single instrument combining objective and subjective measurements.

### 2. BACKGROUND

The human eye is subject to a variety of optical aberrations. Objective determination of low order aberrations such as defocus and astigmatism are commonly measured with an autorefractor. These measurements are used as the basis for a subsequent subjective refraction, using a phoropter, for refinement prior to dispensing a prescription for spectacles or contact lenses, or for use in vision correction surgery such as laser vision correction. An ophthalmic aberrometer, on the other hand, provides a more complete measurement of an eye's optical aberrations. Such measurement is essential for precise aberration correction through customized photorefractive surgery, customized contact lenses or customized intraocular lenses.

An autorefractor is used to produce objective measurement of an eye's defocus power, cylinder power and cylinder axis. However, it is the entire visual system, neural processing and subjective interpretation of perception and processing that determine, in large part, the subject's preferred image or visual experience. A phoropter is used to refine the measurement of the autorefractor through subjective response from the patient. The autorefractor and phoropter are typically stand-alone instruments, and they require different seating and alignment to perform the measurements. Also, a phoropter inserts a number of trial lenses into the viewing path of each subject eye and relies on response from the patient to identify the optimal refractive correction and thus the patient's prescription. Typically, it can take 10 to 30 minutes to determine the final refraction using the autorefractor and phoropter combination.

An ophthalmic aberrometer, i.e. an ophthalmic wavefront instrument, can provide high precision measurement of wavefront aberrations of a subject's eye. The ophthalmic aberrometer is typically an objective instrument and does not provide the subjective refinement needed for refractive surgery, high precision spectacles or contact lenses. When using the autorefractor or the ophthalmic aberrometer, the subject's attention is guided to a nearby internal viewing target that can induce accommodation, and thus what is known as instrument myopia, a shortcoming of objective instruments. In practice, refractive surgeons still commonly rely on the subjective refraction from a phoropter to provide a refined refractive correction, which is time consuming and may be subject to system errors from multiple instruments and multiple operators.

In addition, the trial lenses of the phoropter are at a distance anterior to the cornea of the subject's eye. When refraction is complete, a measurement of this distance (the vertex distance) is taken and the obtained correction needs to be transformed by a calculation to provide the adjusted defocus and astigmatic power correction needed at the position at which the final optical correction is placed or intended, whether for spectacles, contact lenses, photorefractive corneal surgery or for a phakic intraocular lens. Errors may still occur if the vertex distance is not measured accurately with the magnitude of the dioptric power error increasing with increasing refractive power.

### 3. SUMMARY

The present invention contemplates an ophthalmic aberrometer combining measurements of wavefront aberrations and subjective refraction into a single instrument and refers both measurements to the same corneal plane. The present invention also contemplates an ophthalmic aberrometer employing an open-field and subjective correction to overcome instrument myopia and to ensure accurate measurement of the best-corrected visual acuity, in addition to measurement of wavefront aberrations. The present invention further contemplates an ophthalmic aberrometer implementing an optical relay with adjustable optical power compensation to eliminate the need of flipping plurality sets of trial lenses for defocus correction. The present invention also further contemplates an ophthalmic aberrometer making wavefront measurements along a viewing path of the subject eye and enabling accurate measurement of the residual wavefront aberrations after compensating for the subjective refraction.

More specifically, the present invention discloses an ophthalmic aberrometer capable of subjective refraction, comprising:
a first optical relay defining a viewing axis and a working plane of said aberrometer, wherein said first optical relay produces a first conjugated plane of said working plane and wherein said working plane defines a test position for a subject eye;
a second optical relay disposed along said viewing axis and producing a second conjugated plane of said working plane, wherein said first optical relay and second optical relay have collectively a total magnification of one;
a viewing path aligned with said viewing axis and enabling a subject eye to look through said first optical relay and said second optical relay onto a distance viewing chart, wherein said distance viewing chart is located external to the device and positioned meters away from said aberrometer;
a probe beam projected along said viewing axis toward said working plane, wherein said probe beam is of near infrared wavelength;
a dichroic beamsplitter positioned at said viewing axis and behind said second optical relay, wherein said dichroic beamsplitter reflects an infrared beam and transmits visible light, and wherein said dichroic beamsplitter redirects away from said viewing axis a wavefront beam emerging from said working plane;
a defocus adjustment mechanism capable of adjusting at least one of said first and second optical relays to compensate for defocus power of said subject eye, wherein said defocus adjustment mechanism enables said subject eye to focus on said distance viewing chart;
an astigmatism compensator positioned at said first conjugated plane and being adjustable to compensate for the cylindrical error of said subject eye;
an ophthalmic wavefront sensor positioned to receive said wavefront beam and to measure objectively wavefront aberrations of said wavefront beam, wherein said ophthalmic wavefront sensor is located at an optically equivalent position of said first or second conjugated plane and provides measurement data enabling objective adjustment of said defocus adjustment mechanism and said astigmatism compensator;
a pupil camera disposed along said viewing path to monitor pupil position of said subject eye; and
a subjective adjustment mechanism engaging with said defocus adjustment mechanism and enabling feedback from said subject eye to refine said defocus adjustment mechanism;
wherein said ophthalmic aberrometer is capable of measuring wavefront aberrations and also provide a subjective refraction of said subject eye, which looks through said ophthalmic aberrometer and focuses at said distance viewing chart.

Accordingly, a first objective of the present invention is to provide a new and improved ophthalmic aberrometer that combines measurements of wavefront aberrations and subjective refraction in a single instrument and refers both measurements to the same corneal plane.

A second objective of the present invention is to provide a new and improved ophthalmic aberrometer overcoming instrument myopia and enabling measurement of accommodation.

A third objective of the present invention is to provide a new and improved ophthalmic aberrometer employing a power-adjustable optical relay to eliminate the plurality sets of trial lenses for defocus correction.

A fourth objective of the present invention is to provide a new and improved ophthalmic aberrometer providing both precise measurement of wavefront aberrations and subjective refinement of the refractive correction.

In patent publication US2013/0135581, Lai teaches an Integrated Refractor, which integrates objective and subjective refractions into a single instrument. The subjective viewing path of the integrated refractor is adapted in the present invention, and the patent publication US2013/0135581 is thus incorporated into this application by reference.

In the present invention, a specific ophthalmic wavefront sensor is employed to obtain high order aberrations over a large, dilated pupil, which is required for refractive laser surgery. To implement such an ophthalmic wavefront measurement, specific polarizing beam splitters and dichroic optics are integrated to reject surface reflections from relay optics and the subject cornea into the ophthalmic wavefront sensor. In addition, specific illumination and a pupil camera are implemented to ensure precise centration and distance alignment of the subject pupil with respect to the ophthalmic wavefront sensor. The above and other aspects of the present ophthalmic aberrometer thus differentiate clearly the present invention from that of US2013/0135581.

The above and other objectives and advantages of the present invention will become more apparent in the following drawings, detailed description, and claims.

### 4. DRAWINGS

FIG. 1 shows an embodiment of an ophthalmic aberrometer capable of subjective refraction, in accordance with the present invention.

FIG. 2 shows an operating procedure for an ophthalmic aberrometer capable of subjective refraction, in accordance with an embodiment of the present invention.

FIG. 3 shows another embodiment of an ophthalmic aberrometer capable of subjective refraction, in accordance with the present invention.

### 5. DESCRIPTION

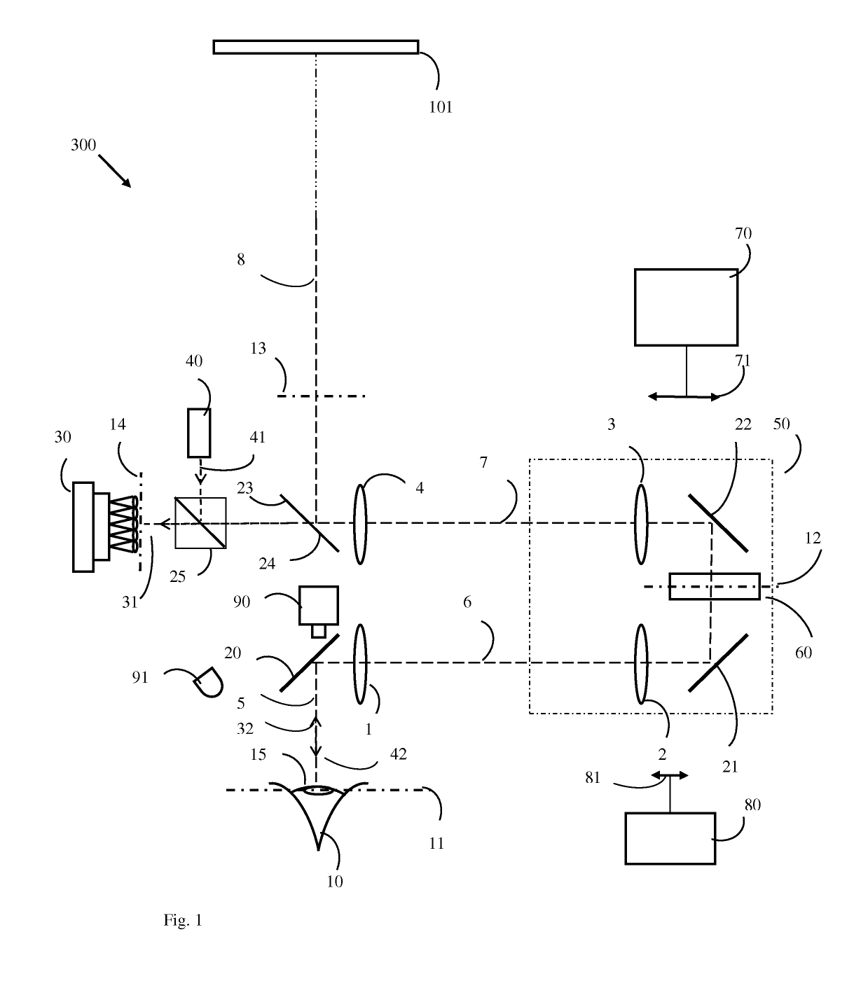

FIG. 1 shows an embodiment of an ophthalmic aberrometer 100 capable of subjective refraction, in accordance with the present invention. The ophthalmic aberrometer 100 consists of a straight viewing path 5 and 8, a first optical trombone with paired lenses 1 and 2, a second optical trombone with paired lenses 3 and 4, turning mirrors 20-23, an astigmatism compensator 60, a dichroic mirror 24, a polarizing beamsplitter 25, a probe beam generator 40, an ophthalmic wavefront sensor 30, a pupil camera 90, a moving stage 50, a defocus adjustment mechanism 70, and a subjective adjustment mechanism 80. A subject eye 10 looks through the viewing path 5 and 8 to fixate on a distance viewing-chart 101.

The first optical trombone with paired lenses 1 and 2 defines a viewing axis 5 and a working plane 11. Viewing axis 5 is overlapped with optical axis 6 via a first turning mirror 20. The first optical trombone 1-2 produces a first conjugated plane 12 of the working plane 11. In a preferred embodiment, the paired lenses 1 and 2 have the same focal length f and thus the first optical trombone 1-2 has an image magnification of −1, i.e., the image through the first optical trombone 1-2 is reversed but has the same size with respect to the object. For practical consideration, the focal length f is preferably about 50 mm to 100 mm, and the paired lenses 1 and 2 are each an achromatic doublet with a diameter of about 0.5 in to 1.0 in.

The second optical trombone with paired lenses 3 and 4 is, in a preferred embodiment, identical to the first optical trombone with paired lenses 1 and 2 and produces a second conjugated plane 13 of the working plane 11. The second optical trombone 3-4 is collinear with the first optical trombone 1-2 via turning mirrors 21 and 22. The first optical trombone 1-2 and the second optical trombone 3-4 thus produce collectively an optical relay of unit magnification between the working plane 11 and the second conjugated plane 13. In this application document, a unit magnification refers to a magnification of +1.

A straight viewing path 5 and 8 is preferably extended straight from the viewing axis 5 to viewing path 8 via turning mirrors 20-23. This straight viewing path 5 and 8 enables subject eye 10 to look through the ophthalmic aberrometer 100 and to fixate on a distance viewing-chart 101. Such a straight viewing path 5 and 8 appears to the subject eye 10 as if it is looking straight through the aberrometer 100 and thus helps to overcome the common effect of instrument myopia. Here and throughout this entire document, including the claims, the response and judgment of the subject eye 10 refers to the collective action of the subject, including the function of the subject's brain.

The first optical trombone 1-2 and the second optical trombone 3-4 are also used to provide optical power adjustment of the viewing path 5-8. As shown in FIG. 1, defocus power of the viewing path 5-8 can be continuously adjusted by adjusting the length of the first and second optical trombones via translating a moving stage 50, which can be controlled via a defocus adjustment mechanism 70 or a subjective adjustment mechanism 80. In a preferred embodiment, the defocus adjustment mechanism 70 is a computer-controlled system that motorizes the moving stage 50 to move along direction 71; and the subjective adjustment mechanism 80 is a manually controlled system that refined adjustment along direction 81 is available to patient himself or an operator. Construction and adjustment of an optical trombone is known to those skilled in the art.

An astigmatism compensator 60 is used to provide astigmatism correction of the viewing path 5-8 and is preferably positioned at the first conjugated plane 12. The astigmatism compensator 60 of FIG. 1 may consist of a set of cylindrical lenses or a pair of positive and negative cylindrical lenses. The astigmatism compensator 60 is preferably motorized via computer control. Astigmatism compensator 60 consisting of a set of cylindrical lenses or a pair of positive and negative cylinder lenses is known to those skilled in the art.

As shown in FIG. 1, a probe beam generator 40 injects a probe beam 41 along the folded viewing path which then impinges as probe beam 42 onto subject eye 10. The probe beam 41 is preferably a low coherent, narrow, high brightness, near infrared light beam, such as a beam from a superluminescent LED. Preferably, the probe beam generator 40 is operated at a wavelength around 780 nm to 830 nm. The probe beam 41 becomes linearly polarized via reflection from polarizing beamsplitter 25.

Turning mirror 23 is also a dichroic mirror 24, i.e. a cold mirror, which reflects visible light and transmits infrared light. The probe beam 41 reflects at polarizing beamsplitter 25, transmits through dichroic mirror 24, travels along optical paths 7-5, and impinges as probe beam 42 into subject pupil 15.

The ophthalmic wavefront sensor 30 is located behind polarizing beamsplitter 25 and receives a wavefront beam 31, which is a reflected beam 32 emerging from the subject pupil 15 and retraces backward the beam path of the probe beam 41 until the polarizing beamsplitter 25. The reflected beam 32 emerging from the subject pupil 15 comprises a polarizing component and a depolarizing (i.e., normal to initial polarization) component. The depolarizing component transmits through the polarizing beamsplitter 25 and becomes the wavefront beam 31. The wavefront beam 31 carries wavefront aberrations of the subject eye 10 plus the wavefront aberrations of all instrument optics, which includes the first optical trombone 1-2, the second optical trombone 3-4, and the astigmatism compensator 60. The ophthalmic wavefront sensor 30 measures and analyzes the wavefront aberrations of the wavefront beam 31 to determine the residual wavefront aberrations of the subject eye 10 after the power compensation through the first and second optical trombones 1-4 and the astigmatism compensation through the astigmatism compensator 60.

As shown in FIG. 1, the ophthalmic wavefront sensor 30 is preferably a Hartman-Shack sensor positioned at a conjugated plane 14, which is optically equivalent to the second conjugated plane 13 of the working plane 11. This way, both the wavefront measurement and the subjective refraction refer to the same working plane 11, and the ophthalmic aberrometer 100 can thus provide more consistent and reliable subjective data on the refraction for use in refractive surgery.

The ophthalmic wavefront sensor 30 is preferably capable of measuring a subject pupil of 6 mm or larger to provide measurement for refractive surgery. An ophthalmic Hartmann-Shack wavefront sensor capable of measuring 8 mm is known to those skilled in the art.

The pupil camera 90 is preferably a video camera and is positioned to view at the eye's pupil 15. An infrared LED 91 is used to illuminate the eye for image capture. The wavelength of the LED 91 is preferably longer than the wavelength of the probe beam 41, e.g. ranging from 840 nm to 940 nm. Illumination with this longer wavelength is long enough to ensure dark dilation of the pupil while still allowing camera resolution and sensitivity.

In a preferable embodiment, high order aberrations of all instrument optics are minimized toward zero through system calibration. High order aberrations in this application refer to third or higher order Zernike polynomials, i.e., aberration terms other than prism, defocus and astigmatism. Thus, the residual wavefront aberrations measured by the wavefront sensor 30 include all the high order aberrations of the subject eye 10.

In a preferable embodiment, defocus compensation via the first and second optical trombones 1-4 is calibrated and readable via a position indicator of the moving stage 50, and the astigmatism compensation via the astigmatism compensator 60 is calibrated and readable via a cylinder power and axial angle indicator of the astigmatism compensator 60. The apparatus and method of position indicator, cylinder power and axial angle indicator are known to those skilled in the art.

The viewing chart 101 is, in a preferred embodiment, placed outside the ophthalmic aberrometer 100 to provide an open view test and to facilitate elimination of instrument myopia. The viewing chart 101 is positioned at a predetermined distance from the subject eye 10. The viewing chart 101 is preferably positioned at an actual optical path length of 20 feet (6 meters) away from the eye 10 for the distance visual acuity test or at a length consistent with distance vision with adjustment of target size as is often done in the clinical situation, and 40 cm away from the second conjugated plane 13 for near visual acuity, though this can in principle be varied for alternative near vision work such as closer for fine detail or further as for computer use or reading sheet music when playing an instrument.

In operation, the subject eye 10 looks through the ophthalmic aberrometer 100 and fixates on the distance viewing-chart 101. The first and second optical trombones 1-4 and the astigmatism 60 are reset to their initial zero position, i.e. zero power in defocus and astigmatism. The ophthalmic wavefront sensor 30 takes a first measurement to determine the initial wavefront aberrations of the subject eye 10 and to calculate objective corrections for the defocus power and the astigmatism (i.e. the cylinder power and axis) of the subject eye 10. A system computer, which is not shown in the figure, drives the defocus adjustment mechanism 70 and the astigmatism compensator 60 to introduce the power and astigmatism compensations into the viewing path 5-8. The ophthalmic wavefront sensor 30 may take another measurement at this point to check whether the residual defocus and astigmatism is about zero and to refine the objective correction. The patient of subject eye 10 can then take a visual acuity test and make his/her subjective refinement on the defocus power correction via the subjective adjustment mechanism 80. Finally, the ophthalmic wavefront sensor 30 measures and records the residual wavefront aberrations and readouts from the moving stage 50 and the astigmatism compensator 60 provide the subjective refraction for the subject eye 10.

In this manner the ophthalmic aberrometer 100 provides a distance viewing chart 101 and enables subjective refinement of the refractive correction, in addition to a wavefront measurement. The ophthalmic aberrometer 100 is thus capable of providing the subjective refraction of the person. Also, the ophthalmic aberrometer 100 makes wavefront measurement along the same viewing path 5-8 of the subject eye 10 and thus ensures accurate measurement of the residual wavefront aberrations after compensating for the subjective refraction.

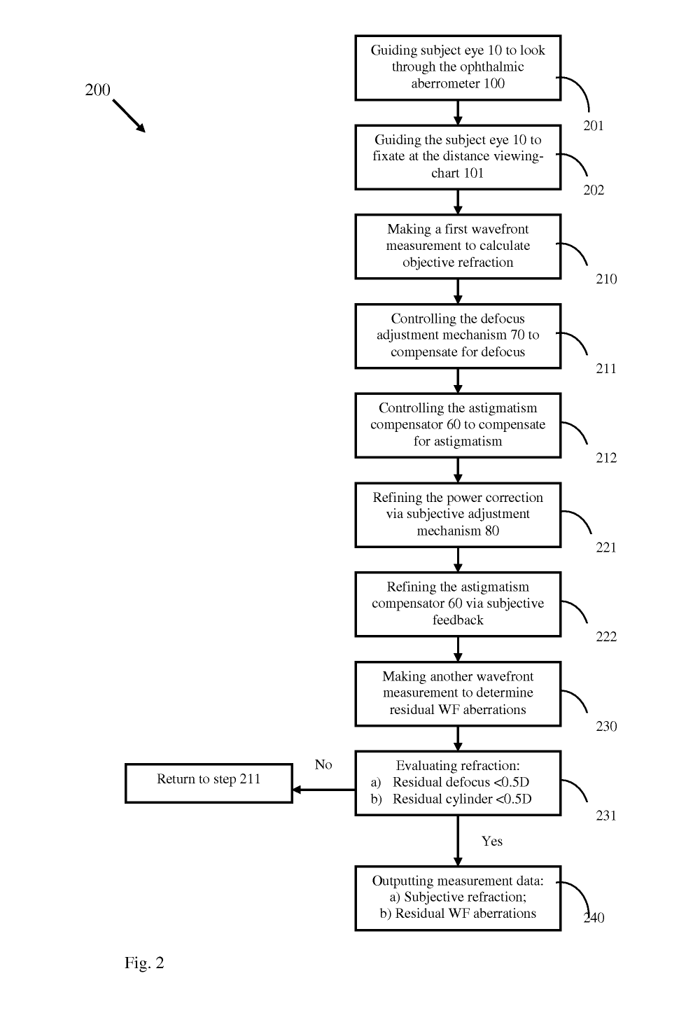

FIG. 2 shows an operation procedure 200 of the ophthalmic aberrometer 100 capable of subjective refraction, in accordance with an embodiment of the present invention. Referring to FIG. 1 and FIG. 2, the operation procedures comprise the following steps:
201) Guiding subject eye 10 to look through the ophthalmic aberrometer 100;
202) Guiding the subject eye 10 to fixate at the distance viewing chart 101;
203) Centering the subject eye 10 with the pupil camera 90;
210) Making a first wavefront measurement with the ophthalmic wavefront sensor 30 to calculate the objective refraction of the subject eye 10;
211) Controlling the defocus adjustment mechanism 70 to compensate for defocus power of the subject eye 10;
212) Controlling the astigmatism compensator 60 to compensate for the astigmatism of subject eye 10;
221) Refining the power correction via subjective adjustment mechanism 80;
222) Refining the astigmatism compensator 60 via subjective feedback;
230) Making another wavefront measurement to determine residual wavefront aberrations;
231) Evaluating the refraction measurement as to whether:
   a) Residual defocusing <0.5 D
   b) Residual cylinder <0.5 D

If the answer to a) and b) is negative, then repeating the procedure from step 211 to confirm the measurement outcome and then moving to the next step;

If the answer to a) and b) is positive, then moving to the next step;
240) Outputting the measurement data:
   a. Subjective refraction based on the readouts of the moving stage 50 and the astigmatism compensator 60
   b. Residual wavefront aberration based on final readout of the ophthalmic wavefront sensor 30

Thus, the measurement procedure 200 provides a distance viewing chart 101 and enables subjective refinement of the refraction in addition to wavefront measurement. The measurement procedure 200 is thus capable of providing a subjective refraction.

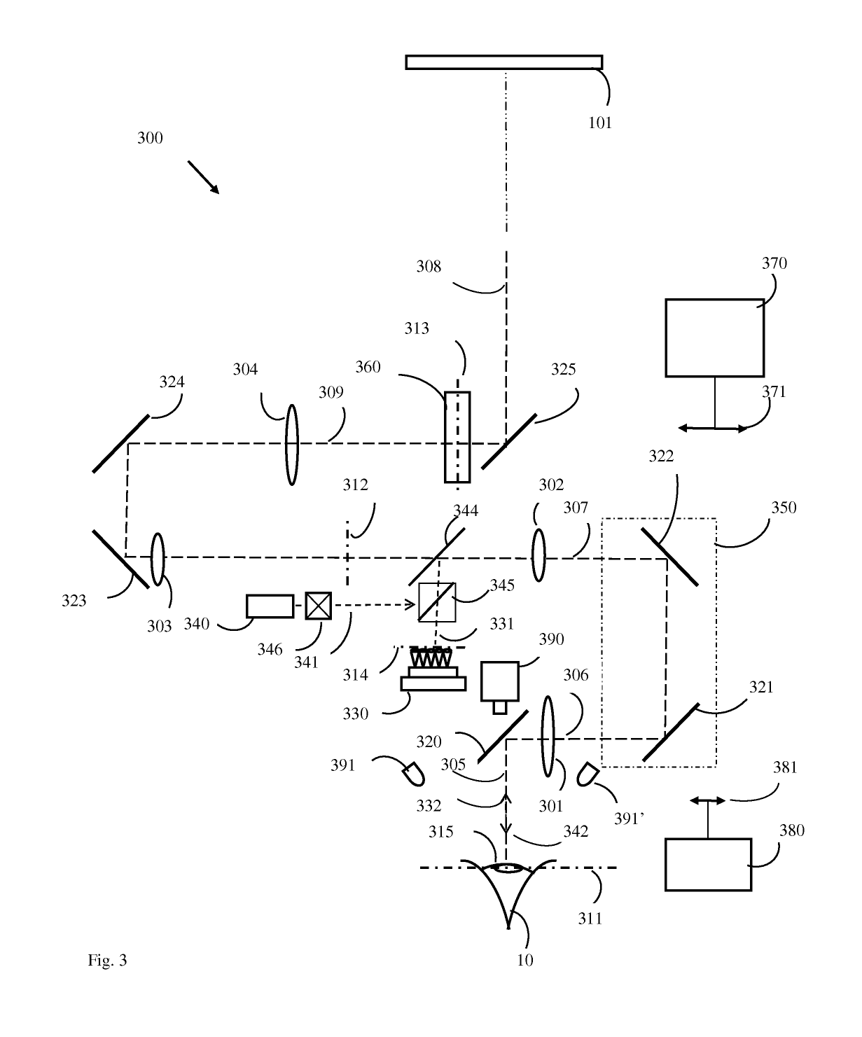

FIG. 3 shows another embodiment of an ophthalmic aberrometer 300 capable of subjective refraction in accordance with the present invention. The ophthalmic aberrometer 300 consists of a straight viewing path 305 and 308, an optical trombone with lenses 301 and 302, an afocal relay with lenses 303 and 304, turning mirrors 320-325, an astigmatism compensator 360, a dichroic mirror 344, a polarizing beamsplitter 345, a probe beam generator 340, an ophthalmic wavefront sensor 330, a pupil camera 390, a moving stage 350, a defocus adjustment mechanism 370, and a subjective adjustment mechanism 380. A subject eye 10 looks through the viewing path 305 and 308 to fixate on a distance viewing chart 101.

The optical trombone with lenses 301 and 302 defines a viewing axis 305 and a working plane 311. Viewing axis 305 is overlapped with optical axis 306 via a first turning mirror 320. The optical trombone 301-302 produces a first conjugated plane 312 of the working plane 311. In another preferred embodiment, the lenses 301 and 302 have respectively focal lengths f1 and f2 and thus the optical trombone 301-302 has an image magnification of −f2/f1, i.e., the image through the optical trombone 301-302 is reversed and the size of the image is f2/f1 with respect to the object. For practical consideration, the focal lengths f1 and f2 are preferably about 50 mm to 100 mm, and the lenses 301 and 302 are each an achromatic doublet with a diameter of about 0.5 in. to 1.0 in.

The second optical relay with lenses 303 and 304 is, in a preferred embodiment, an afocal relay with a reversed magnification of −f1/f2 with respect to the optical trombone 301-302 and produces a second conjugated plane 313 of the working plane 311. The afocal relay 303-304 is collinear with the optical trombone 301-302 via turning mirrors 321-324. The optical trombone 301-302 and the afocal relay 303-304 thus produce collectively an optical relay of unit magnification between the working plane 311 and the second conjugated plane 313.

A viewing path 305 and 308 is preferably extended straight from the viewing axis 305 to viewing path 308 via turning mirrors 320-325. This straight viewing path 305 and 308 enables subject eye 10 to look through the ophthalmic aberrometer 300 and to fixate on a distance viewing chart 101. Such a straight viewing path 305 and 308 appears to the subject eye 10 as if it is looking straight through the aberrometer 300 and thus helps to overcome the common effect of instrument myopia. Here and throughout this entire document, including the claims, the response and judgment of the subject eye 10 refers to the collective action of the subject, including the function of the subject's brain.

The optical trombone 301-302 is also used to provide optical power adjustment of the viewing path 305-308. As shown in FIG. 3, defocus power of the viewing path 305-308 can be continuously adjusted by adjusting the length of the optical trombone via translating a moving stage 350, which can be controlled via a defocus adjustment mechanism 370 or a subjective adjustment mechanism 380. In a preferred embodiment, the defocus adjustment mechanism 370 is a computer-controlled system that motorizes the moving stage 350 to move along direction 371. The subjective adjustment mechanism 380 is a manually controlled system that refines adjustment along direction 381 and is available for use by either the patient or an operator.

An astigmatism compensator 360 is used to provide astigmatism correction of the viewing path 305-308 and is preferably positioned at the second conjugated plane 312. The astigmatism compensator 360 of FIG. 3 may consist of a set of cylindrical lenses or a pair of positive and negative cylindrical lenses. The astigmatism compensator 360 is preferably motorized via computer control. Astigmatism compensator 360 consisting of a set of cylindrical lenses or a pair of positive and negative cylinder lenses is known to those skilled in the art.

As shown in FIG. 3, a probe beam generator 340 injects a probe beam 341, via a dichroic mirror 344, along the folded viewing path which then impinges as probe beam 342 onto subject eye 10. The probe beam 341 is preferably a low coherent, narrow, high brightness, near infrared light beam, such as a beam from a superluminescent LED. Preferably, the probe beam generator 340 is operated at a wavelength around 780 nm to 830 nm. The probe beam 341 becomes linearly polarized via reflection from polarizing beamsplitter 345.

The dichroic mirror 344 is preferably a hot mirror, which reflects infrared light and transmits visible light. The probe beam 341 becomes polarized via a polarizing cube 346 and then reflects at polarizing beamsplitter 345, reflects at dichroic mirror 344, travels along optical paths 307-305, and impinges as probe beam 342 into subject pupil 315. A cross polarization of the polarizing cube 346 and the polarizing beamsplitter 345 enables a purer polarization of the probe beam 342 and thus a better rejection of surface reflections, e.g., from the optics and the subject cornea 10, into the ophthalmic wavefront sensor 330.

The ophthalmic wavefront sensor 330 is located behind polarizing beamsplitter 345 and receives a wavefront beam 331, which is a reflected beam 332 emerging from the subject pupil 315 and retraces backward the beam path of the probe beam 341 until the polarizing beamsplitter 345. The reflected beam 332 emerging from the subject pupil 315 comprises a polarizing component and a depolarizing (i.e., normal to initial polarization) component. The depolarizing component transmits through the polarizing beamsplitter 345 and becomes the wavefront beam 331. The wavefront beam 331 carries wavefront aberrations of the subject eye 10 plus the wavefront aberrations of the trombone optics. The ophthalmic wavefront sensor 330 measures and analyzes the wavefront aberrations of the wavefront beam 331 to determine the residual wavefront aberrations of the subject eye 10 after the power compensation through the optical trombone 301-302.

As shown in FIG. 3, the ophthalmic wavefront sensor 330 is preferably a Hartman-Shack sensor positioned at a conjugated plane 314, which is optically equivalent to the first conjugated plane 312 of the working plane 311. This way, both the wavefront measurement and the subjective refraction refer to the same working plane 311, and the ophthalmic aberrometer 300 can thus provide more consistent and reliable subjective data on the refraction for use in refractive surgery.

The ophthalmic wavefront sensor 330 is preferably capable of measuring a subject pupil of 6 mm or larger to provide a wavefront measurement for refractive surgery. When trombone 301-302 has a magnification f2/f1 smaller than 1, the ophthalmic wavefront sensor 330 is capable of measuring a subject pupil larger than the sensor's aperture. An ophthalmic Hartmann-Shack wavefront sensor capable of measuring 8 mm is known to those skilled in the art.

The pupil camera 390 is preferably a video camera and is positioned to view at the eye's pupil 315. Multiple infrared LEDs 391-391′ are used to illuminate the eye 10 for image capture. The wavelength of the LEDs 391-391′ are preferably longer than the wavelength of the probe beam 341, e.g. ranging from 840 nm to 940 nm. Illumination with this longer wavelength is long enough to ensure dark dilation of the pupil while still allowing camera resolution and sensitivity. The multiple LEDs 391-391′ are preferably installed on a circle around the instrument viewing axis 305, and thus the first Purkinje image of the LEDs 391-391′ (i.e. reflection from the anterior corneal surface) form a circle similar to the image of a keratometer. With the first Purkinje image of LEDs 391-391′, pupil camera 390 can be used to determine precisely the alignment of subject eye 10 with respect to the working plane 311 and the viewing axis 305.

In a preferable embodiment, high order aberrations of the ophthalmic aberrometer 300 are minimized toward zero through system calibration. High order aberrations in this application refer to third or higher order Zernike polynomials, i.e., aberration terms other than prism, defocus and astigmatism. Thus, the residual wavefront aberrations measured by the wavefront sensor 330 include all the high order aberrations of the subject eye 10.

In a preferable embodiment, defocus compensation via the optical trombone 301-302 is calibrated and readable via a position indicator of the moving stage 350, and the astigmatism compensation via the astigmatism compensator 360 is calibrated and readable via a cylinder power and axial angle indicator of the astigmatism compensator 360. The apparatus and method of position indicator, cylinder power and axial angle indicator are known to those skilled in the art.

The viewing chart 101 is, in a preferred embodiment, placed outside the ophthalmic aberrometer 300 to provide an open view test and to facilitate elimination of instrument myopia. The viewing chart 101 is positioned at a predetermined distance from the subject eye 10. The viewing chart 101 is preferably positioned at an actual optical path length of 20 feet (6 meters) away from the eye 10 for the distance visual acuity test or at a length consistent with distance vision with adjustment of target size as is often done in the clinical situation, and 40 cm away from the second conjugated plane 313 for near visual acuity, though this can in principle be varied for alternative near vision work such as closer for fine detail or further as for computer use or reading sheet music when playing an instrument.

In operation, the subject eye 10 looks through the ophthalmic aberrometer 300 and fixates on the distance viewing-chart 101. The optical trombone 301-302 and the astigmatism compensator 360 are reset to their initial zero position, i.e. zero power in defocus and astigmatism. The ophthalmic wavefront sensor 330 takes a first measurement to determine the initial wavefront aberrations of the subject eye 10 and to calculate objective corrections for the defocus power and the astigmatism (i.e. the cylinder power and axis) of the subject eye 10. A system computer, which is not shown in the figure, drives the defocus adjustment mechanism 370 and the astigmatism compensator 360 to introduce the power and astigmatism compensations into the viewing path 305-308. The ophthalmic wavefront sensor 330 may take another measurement at this point to check whether the residual defocus is about zero and to refine the objective correction. The patient of subject eye 10 can then take a visual acuity test and make his/her subjective refinement on the defocus power correction via the subjective adjustment mechanism 380. Finally, the ophthalmic wavefront sensor 330 measures and records the residual wavefront aberrations and readouts from the moving stage 350 and the astigmatism compensator 360 and provides the subjective refraction for the subject eye 10.

In this manner, the ophthalmic aberrometer 300 provides a distance viewing chart 101 and enables subjective refinement of the refractive correction, in addition to a wavefront measurement. The ophthalmic aberrometer 300 is thus capable of providing the subjective refraction of the person. Also, the ophthalmic aberrometer 300 makes a wavefront measurement along the same viewing path 305-308 of the subject eye 10 and thus ensures accurate measurement of the residual wavefront aberrations after compensating for the subjective refraction.

Although the above is described with specific embodiments, various modifications can be made without departing from the scope of the appended claims.

What is claimed is:

1. An ophthalmic aberrometer capable of subjective refraction, comprising:
a viewing path enabling a subject eye to look through and to fixate at/on a distance viewing chart;
a defocus compensator disposed along said viewing path to compensate for defocus error of said subject eye;
an astigmatism compensator disposed along said viewing path to compensate for cylindrical error of said subject eye;
an ophthalmic wavefront sensor disposed along said viewing path to measure objectively the wavefront aberrations of said subject eye, wherein, said ophthalmic wavefront sensor is capable of measuring high order aberrations for a 6 mm pupil or larger;
a pupil camera disposed along said viewing path to monitor pupil position of said subject eye;
an objective adjustment mechanism coupled with said wavefront sensor to drive said defocus compensator and said astigmatism compensator; and
a subjective adjustment mechanism enabling feedback from said subject eye to refine said defocus compensator and said astigmatism compensator;
wherein said ophthalmic aberrometer is capable of measuring wavefront aberrations while also providing a subjective refraction of said subject eye, which looks through said ophthalmic aberrometer and focuses at/on said distance viewing chart.

2. An ophthalmic aberrometer of claim 1, wherein said viewing path consists of an optical relay of unit magnification.

3. An ophthalmic aberrometer of claim 1, wherein said defocus compensator consists of at least an optical trombone.

4. An ophthalmic aberrometer of claim 1, wherein said astigmatism compensator consists of a pair of positive and negative cylindrical lenses.

5. An ophthalmic aberrometer of claim 1, wherein said ophthalmic wavefront sensor consists of a Hartmann-Shack sensor.

6. An ophthalmic aberrometer of claim 1, wherein said ophthalmic wavefront sensor has a measurement diameter of 6 mm or larger.

7. An ophthalmic aberrometer of claim 1, wherein said objective adjustment mechanism is driven with measurement data from said wavefront sensor.

8. An ophthalmic aberrometer of claim 1, wherein said subjective adjustment mechanism is driven with feedback from said subject eye.

9. An ophthalmic aberrometer capable of subjective refraction, comprising:
a first optical relay defining a viewing axis and a working plane of said aberrometer, wherein said first optical relay produces a first conjugated plane of said working plane and wherein said viewing axis and said working plane define a measurement position for a subject eye;
a second optical relay disposed along said viewing axis and producing a second conjugated plane of said working plane, wherein said first optical relay and second optical relay have collectively a total magnification of one;
a viewing path aligned with said viewing axis and enabling said subject eye to look through said first optical relay and said second optical relay onto a distance viewing chart, wherein said distance viewing chart is located external and positioned meters away from said aberrometer;
a probe beam projected along said viewing axis toward said working plane, wherein said probe beam is of near infrared wavelength;
a dichroic beamsplitter positioned at said viewing axis to separate visible light from near infrared of said probe beam and a wavefront beam emerging from said subject eye;
a defocus adjustment mechanism capable of adjusting at least one of said first and second optical relays to compensate for defocus power of said subject eye, wherein said defocus adjustment mechanism enables said subject eye to focus on said distance viewing chart;
an astigmatism compensator positioned at said first or second conjugated plane and being adjustable to compensate for any cylindrical error of said subject eye;
an ophthalmic wavefront sensor positioned to receive said wavefront beam and to measure high order wavefront aberrations for a 6 mm pupil or larger, wherein said ophthalmic wavefront sensor is located at an optical equivalent position of said first or second conjugated plane and provides measurement data enabling objective adjustment of said defocus adjustment mechanism and said astigmatism compensator;
a pupil camera disposed along said viewing path to monitor pupil position of said subject eye; and
a subjective adjustment mechanism engaging with said defocus adjustment mechanism and enabling feedback from said subject eye to refine said defocus adjustment mechanism;
wherein said ophthalmic aberrometer is capable of measuring wavefront aberrations while also providing a subjective refraction of said subject eye, which looks through said ophthalmic aberrometer and focuses at/on said distance viewing chart.

10. An ophthalmic aberrometer of claim 9, wherein at least one of said first optical relay and said second optical relay is a trombone relay.

11. An ophthalmic aberrometer of claim 9, wherein one of said first optical relay and said second optical relay is an afocal relay.

12. An ophthalmic aberrometer of claim 9, wherein one of said first optical relay and said second optical relay is an image-reversing optics.

13. An ophthalmic aberrometer of claim 9, wherein said distance viewing chart is a visual acuity test chart.

14. An ophthalmic aberrometer of claim 9, wherein said probe beam is linearly or circularly polarized.

15. An ophthalmic aberrometer of claim 9, wherein said defocus adjustment mechanism consists of a mechanical translation stage.

16. An ophthalmic aberrometer of claim 9, wherein said defocus adjustment mechanism is driven via a signal from said wavefront sensor.

17. An ophthalmic aberrometer of claim 9, wherein said astigmatism compensator is driven via a signal from said wavefront sensor.

18. An ophthalmic aberrometer of claim 9, wherein said subjective adjustment mechanism consists of a manual adjustment in accordance with feedback from said subject eye.

19. An ophthalmic aberrometer of claim 9, wherein said subjective adjustment mechanism is operable by the patient of said subject eye.

20. A method for measuring ophthalmic aberrations along with subjective refraction, comprising the steps of:
providing a viewing path enabling a subject eye to look through and to fixate at/on a distance viewing chart;
providing a defocus compensator disposed along said viewing path to compensate for a defocus error of said subject eye;
providing an astigmatism compensator disposed along said viewing path to compensate for a cylindrical error of said subject eye;
providing an ophthalmic wavefront sensor disposed along said viewing path to measure objectively wavefront aberrations of said subject eye;
providing a pupil camera disposed along said viewing path to monitor pupil position of said subject eye;
providing an objective adjustment mechanism coupled with said wavefront sensor to drive said defocus compensator and said astigmatism compensator;
providing a subjective adjustment mechanism enabling feedback from said subject eye to refine said defocus compensator and said astigmatism compensator;
measuring wavefront aberrations to calculate initial refractive errors of said subjective eye;
adjusting objectively said defocus compensator to compensate defocus of said calculated refractive errors;
adjusting objectively said astigmatism compensator to compensate astigmatism of said calculated refractive errors;
refining subjectively said defocus compensator to obtain optimal visual acuity of said subject eye; and
measuring residual wavefront aberrations with respect to said defocus compensator and said astigmatism compensator;
wherein said method provides measurements of wavefront aberrations and subjective refraction.
